(19) United States
(12) Patent Application Publication
(10) Pub. No.: US 2006/0283970 A1
(43) Pub. Date: Dec. 21, 2006

(54) Single use air treatment device and method

(76) Inventors: Geoffrey Faires, Cave Creek, AZ (US); Paul Pappalardo, Scottsdale, AZ (US)

Correspondence Address: THE DIAL CORPORATION, 15501 N. DIAL BOULEVARD, SCOTTSDALE, AZ, 85260, US

(21) Appl. No.: 11/151,903

(22) Filed: Jun. 14, 2005

(51) Int. Cl. A61L 9/04

(52) U.S. Cl. 239/6; 239/34; 239/60

(57) ABSTRACT

A fragrance delivery device and method of using it are described. The device has a mold with an inside cavity and an open top, a mold release agent coated on the inside cavity of the mold, a fragranced gel within the mold, and a removable vapor-impermeable lid sealing the open top of the mold.

### FIELD OF THE INVENTION

The present invention relates generally to gel air freshener devices. More particularly, the present invention relates to a small, single use gel air freshener supplied in an inexpensive and disposable mold. The gel air freshener is used by popping the preformed gel shape out from the disposable mold and onto a secondary surface such as onto a plate or into a bowl, or by popping the gel into an area requiring fragrance or malodor control, such as waste baskets, sink drains, garbage disposals, trash cans, trash compactors, cat litter boxes or toilets.

### BACKGROUND OF THE INVENTION

Aqueous-based freestanding gels are a widely used form of air fresheners, used to deliver fragrance and for controlling malodors. Fragrances used in freestanding gel formulations are generally continuously delivered into the air, assisted by the evaporation of the water and additional co-solvents. Representative gel air fresheners currently in the market include Renuzit® Adjustable™ by Dial Corporation, which is a cone-shaped dispenser of gel, and Glade® Secrets™ by S.C. Johnson & Son, which is a much smaller gel air freshener comprising fragrance gel inside a small plastic tray with a foil seal.

These aqueous-based air fresheners are considered “freestanding” in that the gel is solidified to a sufficient mechanical rigidity (gel strength) such that it can stand on its own without deforming in shape. However, aqueous-based air freshener gels are not found without the containers they were cast in. That is, the fragrance gels are designed to scent the room while remaining within the containers they were molded in. In this way, the gel air freshener container functions both as the mold into which the gel is solidified, and as the dispenser from which the fragrance will be dispensed to scent the room. The dispensing container helps keep the fragranced gel from getting on consumers hands and on household surfaces when the product is in use, and for some of the products, helps to maintain a certain level of child-resistance.

For example, the Renuzit® Adjustable™ air freshener is comprised of a gel cast inside a two-piece plastic container that the consumer opens by pulling apart to expose the gel within. The gel remains attached to the base portion and maintains its cast conical shape even though the product is exposed and unsupported around its circumference once the top housing portion is pulled upward. The two-piece plastic housing provides a way for the consumer to control the intensity of the product since there is so much of the gel material present, although it is quite easy to touch the gel when the container is pulled open to any degree. The Glade® Secrets™ by S.C. Johnson & Son is a fragranced gel molded within the cup it is dispensed from. For this product to be used, the consumer peels off an adhesive cover to expose a number of vent slots in the top surface of the cup. The vents are sized small enough that the gel cannot be easily touched but wide enough to allow airflow onto the top surface of the gel and subsequent fragrance release.

A fragranced gel air freshener that must remain within the container that it was molded in leads to many disadvantages for the consumer. One disadvantage of having a gel air freshener anchored within a plastic container is that the gel air freshener will be primarily limited in function to a room air freshener. For example, the product cannot be used to scent and deodorize an in-sink garbage disposal or a toilet unless the gel is literally cut or scooped out from the container prior to use. Furthermore, water-based gel air fresheners shrink quickly around at least one internal surface within the container. This aging of the product reduces the exposed surface area of the product and diminishes its fragrance strength rather quickly. U.S. Pat. No. 5,060,858 by Thomas Santini claims improvement in the continuous delivery of fragrance from an aqueous-based gel air freshener by purposely anchoring the gel into the container to control the direction of shrinkage and the size of the exposed area of the emanating surface.

The present invention is a fragrance delivery device comprising an aqueous-based fragranced gel cast within a very inexpensive and disposable plastic mold, and where the gel is designed to be popped out and freed entirely from its mold prior to use. In this way the product may be used anywhere needed, for example, thrown into trash receptacles, down sink drains, even flushed into toilets. Additionally several gel shapes from various molds may be popped out into a decorative bowl, creating a “potpourri” of fragrances, and an arrangement of colors and decoration, like a fruit bowl. Without the plastic container as a hindrance, an aqueous-based gel air freshener is considerably more versatile than if stuck, permanently anchored, or partially guarded within its plastic container and has more exposed surface area to release fragrance from.

### SUMMARY OF THE INVENTION

This invention generally relates to aqueous-based gel air fresheners cast in plastic molds. In particular, this invention is a gel air freshener cast within a disposable plastic mold of decorative shape. The method of use for this invention is to pop the fragranced gel shape from its disposable mold out onto a secondary surface from which it then liberates fragrance, or out into an environment to be deodorized.

### BRIEF DESCRIPTION OF THE DRAWING FIGURES

The subject invention may be described in conjunction with the appended drawing figures, and where:

FIG. 1 shows a preferred embodiment of the invention.

FIG. 2 shows another preferred embodiment of the invention.

FIG. 3 illustrates a third preferred embodiment of the invention.

FIG. 4 depicts a method of use for one embodiment of the single use air treatment device of the present invention in which the film seal of the mold is peeled away by the consumer to expose a decoratively shaped fragranced gel, and,

FIG. 5 depicts a method of use for one embodiment of the single use air treatment device of the present invention in which the fragranced gel shape is freed from the disposable mold into a local environment.

### DETAILED DESCRIPTION OF EXEMPLARY EMBODIMENTS

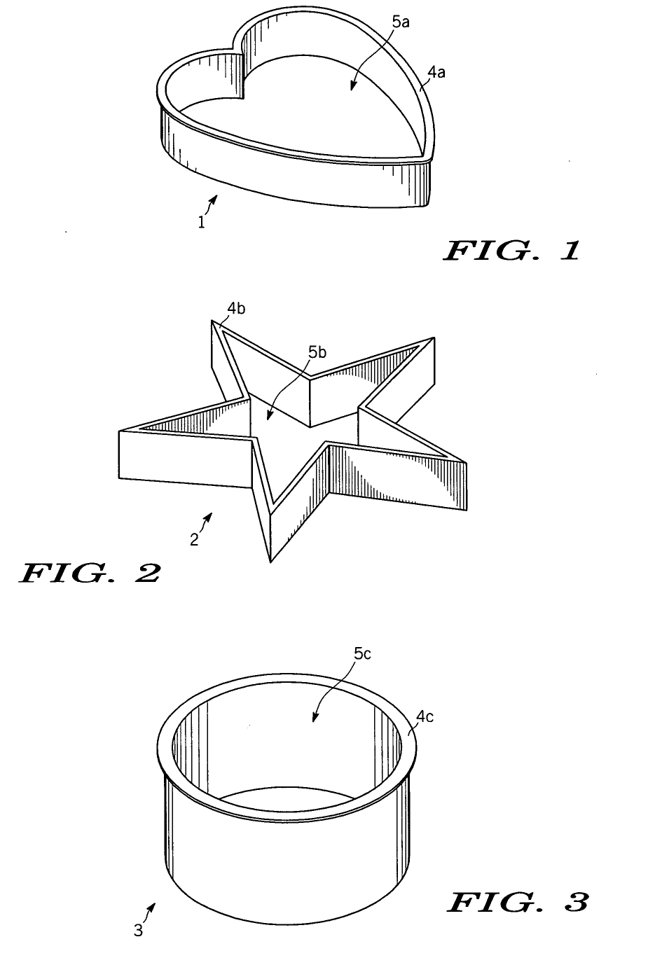

Referring now to FIGS. 1-3, the mold into which the fragrance gel is cast may consist of a variety of shapes and sizes. Similarity is made to candy molds in that this air freshener gel mold may consist of similar decorative and entertaining shapes, for example, a heart shape 1, a star shape 2, a cup shape 3, or other decorative or entertaining fruit, toy, or animal shapes, or various geometrical shapes, and so forth. Within the scope of the present invention, the mold is comprised of from only one to about a few cavities rather than numerous cavities such as found in production food molds designed to repetitively mass-produce the same shape. It is most preferred that the mold functions to cast just one single fragranced gel object, that is, the most preferred mold for this invention comprises a single decorative shaped cavity as demonstrated by the examples in FIGS. 1-3. Being a mold into which a liquid mixture will be poured, it is evident that the top surface will necessarily be flat and planer and the interior cavity will define a decorative shape. Referring to the representative example depicted in FIG. 1, it is preferred that the flat top of the mold comprise a flat lip 4a around a cavity 5a and that this lip measure about 0.12 inch to about 0.50 inch. In FIG. 2, a flat lip 4b comprises the flat top of the mold 2 around a cavity 5b having similar measurements as described above. In FIG. 3 it is preferred that a flat top of the mold 3 comprise a flat lip 4c around a cavity 5c having measurements as described above. Therefore, although any mold within the scope of this invention may have any sort of decorative interior cavity, the top will necessarily be flat, as this is the open top of the mold and it is most preferred that this flat top edge comprise a lip that generally follows the circumference of the cavity of the mold.

The preferred material for the mold in this invention is plastic. Most preferred is polypropylene and the most preferred type of molded plastic for this mold is thermoformed plastic. These preferences allow for a very inexpensive and disposable plastic mold for the fragranced gel. One advantage to the present invention is the reduction in plastic waste for aqueous-based gel air fresheners. Thus it is preferred that the plastic mold be thin thermoformed plastic. Polypropylene plastic is the preferred material because it provides good fragrance resistance and barrier properties.

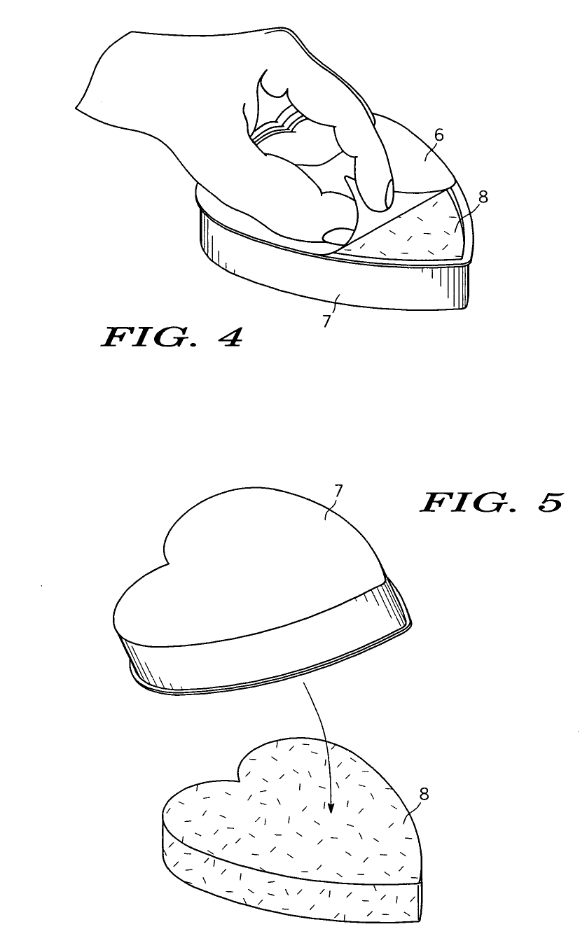

Inside of any of these molds may be poured a hot liquid aqueous fragrance mixture capable of solidifying into a gel upon cooling. After such a process, a lid may be used to seal the mold. Most preferred is that a lid is laid across the open top of the mold and sealingly bonded to the flat top lip of the mold. This type of lid is well known in the food industry and once bonded into place on top of the mold, the product will be sealed much like many food products in the market. Peelable lids of this sort may be comprised of simple plastic films such as polyester, polypropylene, polyamide and the like, or comprised of multiple layer laminates, such as film and foil laminates. Such laminates offer better barrier properties that single layer films. Most preferred for the present invention is to seal the mold that contains the fragranced gel with a plastic film and foil laminate. In this way, the fragranced gel will be better preserved from water and fragrance loss under storage conditions. As practiced in the food industry, the film may be left overhanging one side of the mold such that the consumer can have a spot to grasp it to peel it off from the mold. As shown in FIG. 4, the peelable lid 6 may be grasped and peeled back from mold 7 to expose solid fragrance gel shape 8.

A release agent is coated on the inner cavity of the disposable mold. This is the inner surface that will come in contact with the fragrance gel to be cast within the mold. The release agent, such as a silicone, is used to aid release of the gel from the mold. The present invention is a fragranced gel releasable from its mold. Therefore, a release agent will ensure the cast fragranced gel will easily pop from its mold when desired. Release agent is meant to be any material that reduces the tendency of the gel to adhere to the cavity of the mold. Any type of release agent available to those of ordinary skill in the art may be used in accordance with the present invention. Silicones, lecithin, hydrogenated vegetable oils, or fats, or mixtures thereof may be used as release agents within the scope of the present invention. Most preferred are the silicone spray release agents because they are efficient in very small quantities and will not likely be completely dissolved into the hot aqueous fragrance mixture when this mixture is cast within the coated mold. Furthermore, since the product cast within the mold is a gel air freshener and not an edible food product, any non-edible release agent may be used for these purposes and the silicones are the most readily available. An aerosol spray silicone release agent would be the most preferred release agent for this invention as it also offers speed in use when coating these molds in production.

The fragranced gel within the scope of the present invention is an aqueous-based gel. In general, this type of gel comprises a gelling agent, fragrance, preservative, dyes and water. Preferred gelling agents for this invention are polysaccharides, such as alginates, carboxymethylcellulose, hydroxyethylcellulose, hydroxypropylcellulose, carrageenan, guar gum, locust bean gum, and the like. Most preferred for the present invention is gellation using carrageenan. As is well known in the industry, gellation of aqueous-based air fresheners is preferably achieved using about 2% to about 5% by weight of carrageenan. The fragrance component used in the aqueous-based fragranced gel of the present invention preferably comprises an oil and/or perfume necessary to provide the desired perfume fragrance hedonics and intensity. The fragrance components of the fragranced gel in the present invention comprise one of more volatile organic compounds available from any of the now known, or hereafter established, perfumery suppliers, such as International Flavors and Fragrances (IFF) of New Jersey, Givaudan of New Jersey, Firmenich of New Jersey, etc. Many types of fragrances can be used in the present invention. Preferably the fragrance materials are volatile essential oils. The fragrances, however, may be synthetically derived materials, naturally derived oils, or mixtures thereof. Naturally derived oils include, but are not limited to, bergamot, bitter orange, lemon, mandarin, caraway, cedar leaf, clove oil, cedar wood, geranium, lavender, orange, origanum, white cedar, rose absolute, and the like. Suitable fragrances include but are not limited to fruits, musk, flower scents, herbal scents, woodland scents, derived from pine, spruce, and other forest smells. Fragrances may be derived from various oils such as the foregoing essential oils, or from plant materials such as peppermint, spearmint, and the like.

The aqueous-based gel of the present invention requires a preservative to prevent microbial growth such as mold growth. Preferred preservatives are of the oxazolidinone family of preservatives, most preferably Neolone or Kathon from Rohm & Hass. These types of preservatives are used from about 0.01% to about 0.1% by weight to prevent microbial growth on the fragranced gel.

Additionally it is preferred that the aqueous-based gel of the present invention also comprises colorants. Any water-soluble dyes known to those skilled in the art may be incorporated into the fragranced gel to add aesthetic appeal. Mixtures of colorants may provide a spectrum of color choices for the gel and most preferred are a variety of colors to coordinate with the various mold shapes and fragrance variants.

The remainder of the fragranced gel within the present invention comprises water. A hot aqueous mixture of polysaccharide gelling agent, fragrance, preservative and dyes, with some ingredients suspended and some dissolved, is poured into the mold. Upon cooling to ambient temperature the liquid will solidify into a rigid structure. The shape will have sufficient rigidity such that it can be successfully popped out from the mold into which it was cast without damage to the shape and aesthetic appeal.

The fragrance delivery device of the present invention may find many uses for scenting and deodorizing spaces within a home. The method of use for this invention is to free the fragranced gel shape entirely from its mold prior to use. Referring again to FIG. 4, the consumer is expected to peel away and discard the film lid 6 sealed on the mold 7, then, as shown in FIG. 5, invert and apply pressure to the deformable plastic mold 7 to pop the gel shape 8 from the mold 7 to the desired location, and then to discard and not reuse the mold.

The fragranced gel entirely freed from its mold has many uses. It may be placed on a plate or in a bowl. Fragranced gel of many different colors, fragrances and shapes may be placed within a bowl to create a decorative menagerie of fragrances and colors. In this manner, a bowl full of decorative fragranced gel shapes resembles potpourri. Gel shapes freed from their molds and placed on a plate or in a bowl will provide fragrance for a room space. The more gel shapes that are put out into a room, the more intense the overall fragrance will be.

Additionally, the fragranced gel of the present invention may be ground up in an in-sink garbage disposal to deodorize the garbage disposal, or may be placed inside a sink drain. Or the fragranced gel may be popped from its mold into a wastebasket or other trash receptacle to deodorize the receptacle, or it may be dropped into the toilet water within the bowl of a toilet. Alternatively, the fragranced gel may be popped from its mold and placed into the tank of a toilet to discretely fragrance the toilet and the area surrounding it.

We claim:

1. A fragrance delivery device comprising,
a. a mold with inside cavity and open top,
b. a mold release agent coated on the inside cavity of said mold,
c. a fragranced gel within said mold, and,
d. a removable vapor-impermeable lid sealing open top of said mold.

2. The device of claim 1, wherein said mold is plastic.

3. The device of claim 2, wherein said lid is comprised of a film and foil laminate.

4. The device of claim 3, wherein said fragranced gel comprises,
a. a gelling agent,
b. a fragrance,
c. a preservative,
d. a colorant, and,
e. water.

5. The device of claim 1, wherein said mold release agent comprises a silicone.

6. The device of claim 1, wherein said mold comprises a decorative shape.

7. A method for treating an environment with fragrance comprising,
a. supplying a fragrance delivery device comprising a disposable mold with a removable lid and a fragranced gel contained within and releasable from said mold, and,
b. removing said lid from said mold, popping said fragranced gel out from said mold into said environment.

8. A method for counteracting the odors within a trash receptacle comprising,
a. supplying a fragrance delivery device comprising a disposable mold with removable lid and a fragranced gel contained within and releasable from said mold, and,
b. removing said lid from said mold and popping said gel out from said mold into said trash receptacle.

9. A method for counteracting the odors within a sink drain comprising,
a. supplying a fragrance delivery device comprising a disposable mold with a removable lid and a fragranced gel contained within and releasable from said mold, and,
b. removing said lid from said mold and popping said gel out from said mold into said sink drain.

10. A method for counteracting odors within a trash compactor comprising,
a. supplying a fragrance delivery device comprising a disposable mold with a removable lid and a fragranced gel contained within and releasable from said mold, and,
b. removing said lid from said mold and popping said gel out from said mold into said trash compactor.

11. A method for counteracting the odors within and surrounding a toilet comprising,
a. supplying a fragrance delivery device comprising a disposable mold with a removable lid and a fragranced gel contained within and releasable from said mold, and,
b. removing said lid from said mold and popping said gel out from said mold into said toilet.

12. A method for assembling a decorative bowl of fragranced gel shapes comprising,
a. supplying more than one fragrance delivery device, each comprising a decoratively shaped mold with removable lid and a fragranced and colored gel contained within and releasable from said mold, and,
b. removing said lid from each mold and popping each gel out from said mold into a decorative bowl to create a mixture of fragranced gel shapes.
